(19) United States
(12) Patent Application Publication
(10) Pub. No.: US 2021/0007330 A1
(43) Pub. Date: Jan. 14, 2021

(54) SYSTEM AND METHOD FOR DETERMINING ANIMAL BEHAVIORAL PHENOTYPES

(71) Applicant: GROWSAFE SYSTEMS LTD., Calgary (CA)

(72) Inventors: Camiel HUISMA, Airdrie (CA); Alison SUNSTRUM, Airdrie (CA)

(21) Appl. No.: 16/980,072

(22) PCT Filed: Mar. 13, 2019
(86) PCT No.: PCT/IB2019/000247

§ 371 Date: Sep. 11, 2020

Related U.S. Application Data
(60) Provisional application No. 62/642,109, filed on Mar. 13, 2018.

(51) Int. Cl. A01K 29/00; A61D 19/02; A61D 19/04; A01K 5/01; A01K 7/00; G06Q 10/04; G06Q 50/02; G16H 40/67; G16H 20/60; G16H 50/70; G06Q 10/06; G01G 17/08

(52) U.S. Cl. CPC A01K 29/005; A61D 19/02; A61D 19/04; A01K 5/01; A01K 7/00; G01G 17/08; G06Q 50/02; G16H 40/67; G16H 20/60; G16H 50/70; G06Q 10/06315; G06Q 10/04

(57) ABSTRACT

A highly automated system and method for predicting, identifying and quantifying unique phenotypes of an animal that are beneficial for enhancing the performance, well-being and profitability of animals in a given production environment based on analysis of data from high frequency weight measurements of a feed.

### FIELD OF THE INVENTION

The present invention relates to a highly automated system and method for predicting and identifying unique phenotypes of an animal that are beneficial for enhancing the performance, well-being and production profitability of animals in a given production environment

### BACKGROUND OF THE INVENTION

The ability to identify animals with superior health and performance attributes for breeding purposes has largely been focused on traits that can be visually identified and simply recorded. A few gross measurements over an animal's lifetime may be taken and statistical methods are then used to estimate the likely performance of its progeny. Although significant advancement in livestock performance, particularly in growth has been made using this method, several antagonisms to the trait of interest have been experienced with negative outcome. In terms of animals with long generational intervals, such as cattle, the ability to collect sufficient data to better inform these estimates is difficult and often hampered by the long-time periods required to evaluate accuracy of data. To utilize this type of breeding information requires the comparison of animals to contemporaries tested in similar conditions and usually requires a large comparative population collected over decades. This data typically needs to be assembled by a third party such as a breed association or University research center with the associated complexity of data management, cost and reporting time delay.

Recent developments in semi-automated and electronic data collection has enabled collection of data to report measurements of traits such as residual feed intake collected primarily in research environments. Residual feed intake, a phenotypic measurement of feed efficiency, was first identified by researchers in the 1960's.

With the sequencing of the bovine genome the possibility to collect and use genotypes to identify gross animal traits of economic relevance has been offered as a potential solution to better predict genetic merit and future progeny performance. A genotype is the set of genes in an animal's DNA which is responsible for a particular trait. A phenotype is the physical expression, or characteristics, of that trait. After many years of research it has been determined that genomic predictors alone may not provide sufficient accurate information to make informed breeding decisions.

Farmers and ranchers can see gross behaviors in animal and have long inferred both positive and negative behavioral attributes to their animals. Few have selected animals for these traits. The complexity of objectively defining behavior has limited its use in formal genetic selection programs. The science of animal behavior has largely been limited to gross behaviors that can be visually seen and heard.

Genetics has been thought to play a large role in the progress of a herd, flock, or group of animals. To genetically enhance a population, herd, troop, flock, or group of animals, the best individual animals were selected to use as breeding stock based on predictions of the superior performance of offspring that were yet to be born. These predictions were generally an estimate of genetic merit based on the use of statistical analysis of performance or phenotypic data of an individual animal and its progenitors. This is a well-accepted procedure and is the basis of genetic improvement schemes for several types of animals.

The genetic merit of an individual generally relies entirely on the data of relatives of that individual. A lack of information of individual animals within a population, herd, troop, flock, or group of animals at an early stage reduces the ability to make decisions about the potential future use of such individuals especially with respect to their usefulness in breeding strategies. Consequently the rate of genetic gain of desired biological or performance traits of the animal group under selection is less than that which would be achievable with such data.

A primary purpose of genetic breeding programs is to pass on desired biologic or performance traits in the genes of the selected breeding individual to its offspring. Although technology allows for the selection and transfer of specific genes to an offspring, there have been difficulties in predicting the correlation between genes and the desired phenotypic traits. Since establishing a population, herd, troop, flock, or group of animals having enhanced genetics, by breeding genetically superior individuals, has been inefficient and not as successful as once predicted to be, attention has shifted to other means to achieve the desired result. There has been a focus of attention on defining behavioral phenotypes which determine the state or well-being of an animal. By determining beneficial behavioral phenotypes, individual animals, exhibiting the desired phenotypic trait(s), can be detected and utilized in breeding programs for enhancing the production and profitability of animals.

Generally the process of identifying behavioral phenotypes is based on a long term tracking of an animal's state or well-being and observed behaviors of that individual animal. After an extended period of time, the observed behaviors of a selection of animals that were determined to be healthier or have a greater well-being were considered. Given the selection of individual animals, attempts were made to identify observed behavioral phenotypes that were common to the individual animals within the group. Although identifying behavioral phenotypes which were though to correlate with the state or well-being of an animal may have been beneficial in selecting animals to participate in a breeding program, the process of gathering data was often imprecise thus leading to inaccurate determinations of the health or well-being of an animal or inaccurate identification of animal behaviors. These methods often fail to accurately identify and/or quantify certain behavioral phenotypes that may have a greater impact on the state or well-being of an animal than other phenotypes. Additionally, the know methods have been unable to correlate behavioral phenotypes or combinations of behavioral phenotypes with specific environmental factors For example, one phenotype may be beneficial for an animal in a production area in one environment, but the same phenotype may be detrimental for the same animal in a production area of a different environment.

### SUMMARY OF THE INVENTION

Wherefore, it is an object of the present invention to overcome the above-mentioned shortcomings and drawbacks associated with the prior art breeding techniques by accurately identifying one or more behavioral phenotypes that have an impact on the state or well-being of an animal.

Another object of the invention is to provide a system and a method for predicting with a specified degree of confidence, and determining unique phenotypes of an animal that are beneficial for enhancing the productivity and therefore profitability of animals in a given environment. The present invention is directed at a system and method which facilitates determining and defining a multiple of phenotypes of a desired animal based on collected data, and determining the probability of transition from a state that may not be visually apparent but digitally identifiable.

Another object of the present invention is to provide a system and a method for predicting with a specified degree of confidence unique animal behavioral phenotypes that are effective in determining the current and future state and well-being of an animal. The system and the method include acquiring and monitoring animal consumption, growth and other behavioral data for a number of animals over a defined period of time, building a scaling multi-dimensional probability matrix that uses past phenotypic and genomic data to inform future predictions and in order to define animal behavioral phenotypes which, if possessed by an animal, leads to an animal that (1) is healthier, (2) has a greater well-being, and (3) can withstand environmental and other stressors, (4) can better adapt to changes in the quality, quantity and type of feed provided for consumption, i.e., diet adaptation, and/or can be specified for market categorization such as absence or prevalence of certain pharmaceutical treatment such as antibiotics.

It is further beneficial to identify desired animal behavioral phenotypes based on the collected consumption and behavioral data and taking into consideration animal genotypes as well as the animal's production environment. By utilizing animals of a desired genotype with desired physical phenotypes and possessing desired behavioral phenotypes in a breeding program, the population of animals having the desired genotype and phenotypes increases and enhances the production and therefor profitability of animals. The system and method also enables matching animals to production environments or in other words correlating defined beneficial animal behavioral phenotypes with specific animal production environments, such that animal production facilities having a certain production environment, e.g., climate condition, soil condition, or terrain features, can stock and/or purchase young animals having a specific genotype exhibiting the desired behavioral phenotypes, thereby enhancing the production and profitability of animals.

In the method for defining behavioral phenotypes, the physiology of the animal such as its body weight, growth, feed and water intake activity, as well as its interaction with other animals within the population, herd, troop, flock or group and its response to changes are generally indicative of the state and well-being of the particular animal. Since behavioral phenotypes can have an impact on the state and well-being of an animal, by correlating animal behavioral phenotypes with animals that are considered as being successful and productive, e.g., healthy animals having a high feed efficiency, it can be predicted that animals having specific behavioral phenotypes will more likely result in the production of successful animals. With the system and the method according to the invention, variations of an animal's physiology are numerically defined. A sufficient narrowing of this variation will bring about different states of the animal. By matching up successful outcomes of animal production to treatments and different states of the animal, it is possible to determine the right amount of narrowing of the variation for it to have practical value. The main fundamentals of determining the physiology or rather the variations of physiology of the animal reside in the collection and analysis of different types of data. With regard to this, it should be recognized that the amount and diversity of collected data are vital to better defining advantageous behavioral phenotypes.

One example of an advantageous behavioral phenotype is referred to as residual feed intake which enumerates the feed efficiency trait of a particular animal. This trait is the difference between an animal's measured feed intake and water intake and the animal's expected feed requirements for growth and maintenance given the animal's body weight and performance. An efficient animal eats less feed than expected based on the animal's body weight and performance. Breeding highly efficient animals can have a significant impact on the overall efficiency of the group of animals and its progeny performance. It has been found that in a group of animals having high feed efficiency traits, the group can include a larger number of individuals while consuming the same amount of feed as a group of animals having average feed efficiency traits. Breeding animals having high feed efficiency traits has led to an increase in the number of individual animals in the group that possess this beneficial trait. Calculating the residual feed intake and thus enumerating the feed efficiency trait of an animal requires periodically measuring the actual feed intake and water intake of the animal.

The subject matter of U.S. Pat. Nos. 6,868,804 and 8,930,148 includes systems and methods which are known to identify, measure, monitor and manage the consumption behavior, substance intake, body weight and growth of individual animals in their usual production environment including range, pasture, feedlot, dairy and farm without disruption to typical behaviors in order to determine, analyze, model and predict a variety of conditions relating to animal health, productivity, efficiency and quality. The entire subject matter of both of these patents, i.e., U.S. Pat. Nos. 6,868,804 and 8,930,148 are fully incorporated herein by reference thereto.

In these systems, an identification transmitter is attached to each individual animal. When an animal approaches a feeding station, an antenna, associated with the feeding station, receives an identification signal from the transmitter which identifies the specific individual animal feeding at the trough of the feeding station. While at the feeding station, one weighing device measures the animal's weight and other weighing devices measure the weight of the feed in the feeding trough. The animal identification signal and weight measurements are transmitted to a computer which records and analyses the collected data. From the collected and analyzed data over a period of time, the computer can then determine and monitor an animal's weight and gain, growth rate and the weight of feed/water consumed, e.g., feed/water intake by the animal over a period of time. Ultimately the weight data can be used to determine, among others purposes described below, the residual feed intake and the feed/water retention of an animal. Hereinafter, feed/water retention is defined as a duration of time over which feed and/or water particles, consumed by the animal, remain inside the animal and contributes to the growth of the animal. The feed/water retention is understood to have an impact on the digestibility of the consumed feed/water as well as the amount of methane gas produced by the animal during digestion of the consumed feed/water. Based on the determined values of factors such as residual feed intake and feed/water retention, the system can model and predict an animal's health and growth, performance, feed utilization, manure and methane output.

High frequency collection of a variety of associated data and measurements is fundamental to the process of accurately defining animal behavioral phenotypes. Advantageous data and measurements to be collected, according to the method and system of the invention, typically include: trough weight data consisting of a trough identifier, a time stamp and a weight; body weight data consisting of a scale identifier, a time stamp and a body weight; and behavior data consisting of an animal identifier, a time stamp and a location identifier. The location identifier typically relates to the trough, but multiple antennas could be located in the trough allowing the method and system to determine a head location within the trough. Further beneficial data to be collected relate to the production environment and can include measurements of the temperature, humidity, precipitation, wind speed and barometric pressure, just to name a few examples. These environmental measurements can also include a time stamp such that these measurements can be correlated to the other data described above. It is to be understood that the above noted data should be collected as frequently as possible. Preferably data/measurements should be collected continuously by the method and system. The increased amount of collected measurements allows for an increased resolution in the measurements taken which, in turn, leads to an increase in the accuracy of the measurements.

Another benefit of the high frequency collection of measurements of, for example, feed trough weights or partial body weights using weighing platforms as described in U.S. Pat. Nos. 6,868,804 and 8,930,148 and which are generally utilized to classify the state of an animal. From such measurements, an animal can be recognized as healthy, gaining, and finished and within these, as in the case of disease, may be able to determine whether an animal is in a state of sub-clinical or clinical disease. Based on its state classification, an animal may be kept in its original location for continued weight gain, separated from the group to proceed to market or processing, isolated from the group for treatment of the disease or some other type of intervention. The state of an animal may be predicted by the animal's residual feed intake, which requires accurate measurements of data such as the weight of feed intake and the weight gain of the animal.

It should be recognized that desirable traits in animal production relate, in one way or another, to animal behavior. In addition, the desirability of a trait or behavioral phenotype can often depend on a variety of factors. For example, the trait of being aggressive may be beneficial for an animal in a production environment where feed is less plentiful or where the temperatures are generally colder. As an animal having the trait of being aggressive tends to be higher in the order in which animals of a group eat, the assumption is that aggressive animals will access feed first and consume as much feed as desired and thus will be well nourished and have a sufficient amount energy to stay warm in a colder environment. Whereas, in a generally warmer environment having relatively higher temperatures, aggressive animals may become subject to heat stress due to exerting excessive amounts of energy while defending their territory. Since the determination of whether a specific trait or behavior, e.g., highly aggressive, is beneficial or not for a particular animal may depend on the growing or production environment, it is advantageous to collect production environment related data, e.g., temperature, precipitation and humidity, hours of daylight/darkness, the type of terrain, e.g., hilly or flat terrain, for the production environment.

As noted above, the more data collected over time, the better and more accurate the results. As such, a primary objective of the invention is the substantially continuous collection of data or the collection of data at a high frequency rate. The increase in data collection results in an increased accuracy of the measure of an individual animal's feed intake, especially when collected under adverse environment conditions such as extreme temperatures, wind, rain, snow as well as other less predictable events, such as other the daily activity of the animal as well as the amount that the animal feeding away of the troughs.

Therefore another object of the invention is to collect and analyze a variety of identification data, time stamp data, weight data, and environmental data more frequently to more accurately define behavioral phenotypes in animals and to identify individual animals having the defined beneficial phenotypes. With more frequent collection of different types of data and by analyzing this data, it is possible to more accurately define behavioral phenotypes and determine the state or well-being of a particular animal. By continuously collecting and analyzing data, it is possible to detect when the state of an animal changes and determine from and into which state the animals changes, e.g., from healthy to unhealthy or from unhealthy to healthy. By analysis of the weight and behavioral data, it is possible to define behavioral phenotypes that identify: which animal feeds first after the trough is supplied with feed; which animal consumes more feed when the trough is relatively full; which animal consumes what part of its feed intake at what time of day; how much an animal routs through the feed searching for specific feed particles, i.e., feed sorting; and which animal is more dominant as determined by which animal(s) pushes other animals out of the bunk and during what period of the day. Feed sorting is to be understood as the behavior of animals to consume specific “desired” feed particles and avoid other “less desired feed particles while consuming mixed rations. For example, animals will often consume greater amounts of highly-fermentable carbohydrates than longer forage, high fiber particles. This behavior can influence the animal's nutrient intake and reduces the nutritive value of the feed left in the bunk, thereby also affecting the nutrient intake of animals feeding subsequently. The weight and behavioral data can further be manipulated to define a behavioral phenotype which determines an individual's feeding pattern including time of feeding event, bite duration, bite frequency, bite pressure and/or bite pressure variation.

It is also an object of the present invention to correlate beneficial animal genotypes and behavioral phenotypes with specific production environments. In other words, given a certain production environment, it is an object of the method and system of the invention to determine which animal genotype(s) and animal behavioral phenotype(s) should an animal possess in that certain production environment in order to yield a highly successful product, i.e., typically have the highest feed efficiency so that the animal grows as fast as possibly while consuming the least amount of feed and water. Based on the determined correlation between behavioral phenotypes and production environments, animals identified by genotype(s) and as having beneficial behavioral phenotypes can then be utilized, for the local breeding programs so as to maximize the population of animals in the group which have these identified desired traits for the local environment. Further based on the correlation between the behavioral phenotypes and local production environments, animal producers can focus on introducing young animals into the group that are of a certain genotype(s) and possess the one or more behavioral phenotypes that are best suited to animals in the given local production environment, such that the individual animals forming the group have high feed efficiencies and thus yield a highly successful product.

The present invention also relates to a method for defining animal behavioral phenotypes which, in a specific environment, are beneficial for the physiology of an animal located within the specific environment. The method comprises the steps of: collecting consumption data and weight gain data for the animal over a period of time; collecting animal behavioral data of the animal over the period of time; identifying a genotype of the animal; analyzing and manipulating the consumption data, the weight gain data and the behavioral data of the animal to define a positive behavioral phenotype that correlates to a high state of the physiology of the animal that is greater than the physiology of an animal of the same genotype and not possessing the positive behavioral phenotype; identifying other animals of the same genotype and possessing the positive behavioral phenotype; and forming a group of animals from the animal and the other animals having the same genotype and possessing the positive behavioral phenotype to produce animals having a high physiology that is greater than a group of animals of the same genotype and not possessing the positive behavioral phenotype.

The present invention also relates to a method of at least one of identifying, defining and quantifying a behavioral phenotype from high frequency weight measurements of a feed trough visited by an individual animal. The behavioral phenotype describing at least one of an animal behavior and a behavioral response of an animal to a condition.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. The invention will now be described, by way of example, with reference to the accompanying drawings in which:

FIG. 1 is a diagrammatic perspective view of a single measurement unit of the system for measuring the weight of an animal in accordance with the teachings of the invention;

FIG. 1A is a diagrammatic perspective view of the system having multiple measurement units for measuring the weight of multiple animals in accordance with the teachings of the invention;

FIG. 1B is a diagrammatic perspective view of a measurement unit for measuring the weight of feed troughs of the method and system in accordance with the teachings of the invention;

FIG. 2 is a diagrammatic schematic representation showing details of various components of the system and the method in accordance with the teachings of the invention;

FIG. 3 is a diagrammatic graphic illustration showing a growth curve for an animal based upon measured feed and water intake weights over a period of time;

FIG. 4 is a diagrammatic graphic illustration showing averaged retention curves of feed and water consumed by an animal over the course of different feeding and drinking events;

FIG. 5 is a diagrammatic graphic illustration showing an averaged retention curve for an animal;

FIG. 6 is a diagram illustrating a linear regression run on filtered weight-time data for a feeding event in accordance with the teachings of the invention;

FIG. 7 is a diagrammatic graphic illustration showing an average behavior intensity for determining a who feeds first rank of an animal;

FIG. 8 is a diagrammatic graphic illustration of data for determining a who feeds first rank of an animal;

FIG. 9 is a diagrammatic graphic illustration showing an analysis of animal feeding rates during a period of high competition for feed and a period for low competition for feed;

FIG. 10 is a diagrammatic graphic illustration showing an analysis of animal feeding rates during period of high competition for feed and during period of low competition for feed;

FIG. 11 is a diagrammatic graphic illustration showing an analysis of animal feeding rates for different animals at a feed trough over a period of time;

FIG. 12 is a diagrammatic graphic illustration showing the determination of bite pressure, bite duration and bite frequency for an animal;

FIG. 13 is a diagrammatic graphic illustration showing the determination of empty bunk attendance;

FIG. 14 is a diagrammatic graphic illustration showing analysis related to feed sorting;

FIG. 15 is a diagrammatic graphic illustration showing analysis and determination of the flightiness of an animal; and

FIGS. 16A, 16B are diagrammatic illustrations of animals having different body shapes.

It should be understood that the drawings are not necessarily to scale and that the disclosed embodiments are sometimes illustrated diagrammatical and in partial views. In certain instances, details which are not necessary for an understanding of this disclosure or which render other details difficult to perceive may have been omitted. It should be understood, of course, that this disclosure is not limited to the particular embodiments illustrated herein.

### DETAILED DESCRIPTION OF THE PREFERRED EMBODIMENTS

The present invention will be understood by reference to the following detailed description, which should be read in conjunction with the appended drawings. It is to be appreciated that the following detailed description of various embodiments is by way of example only and is not meant to limit, in any way, the scope of the present invention.

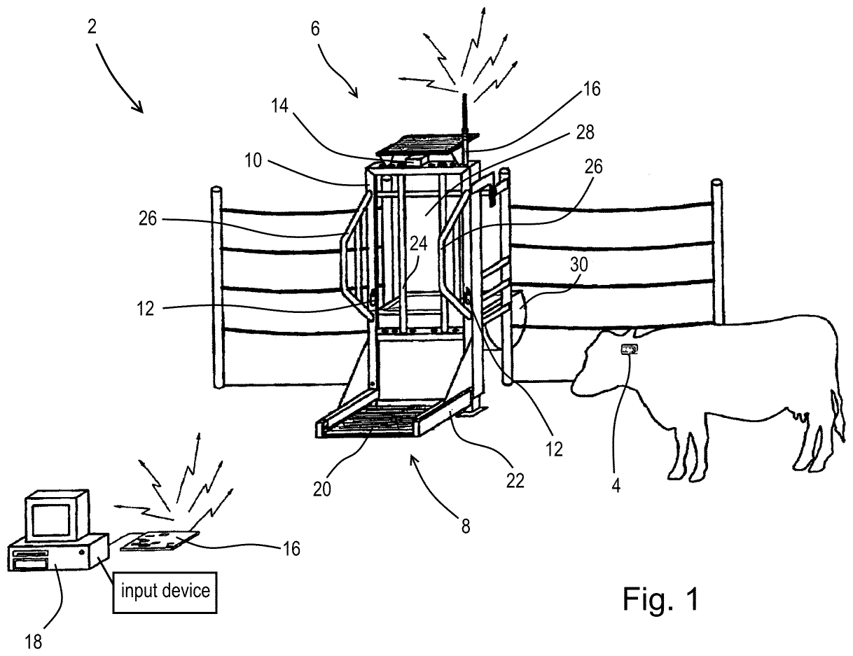

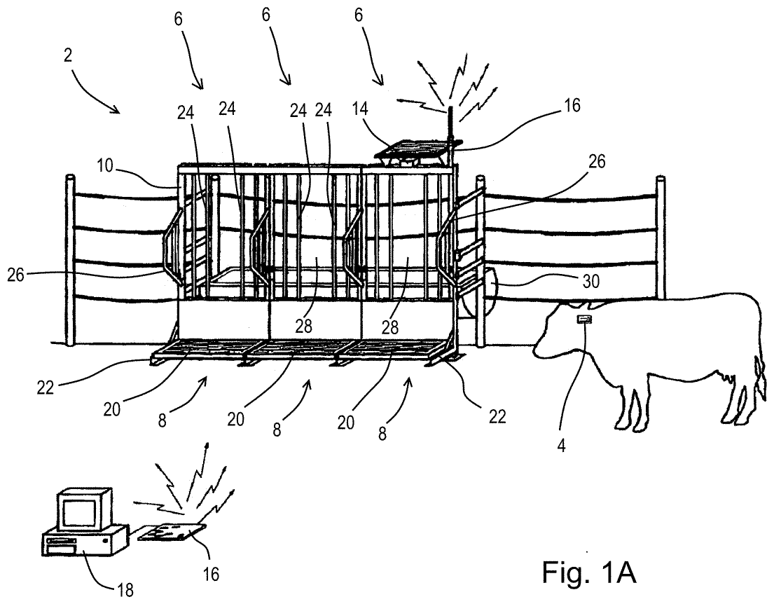

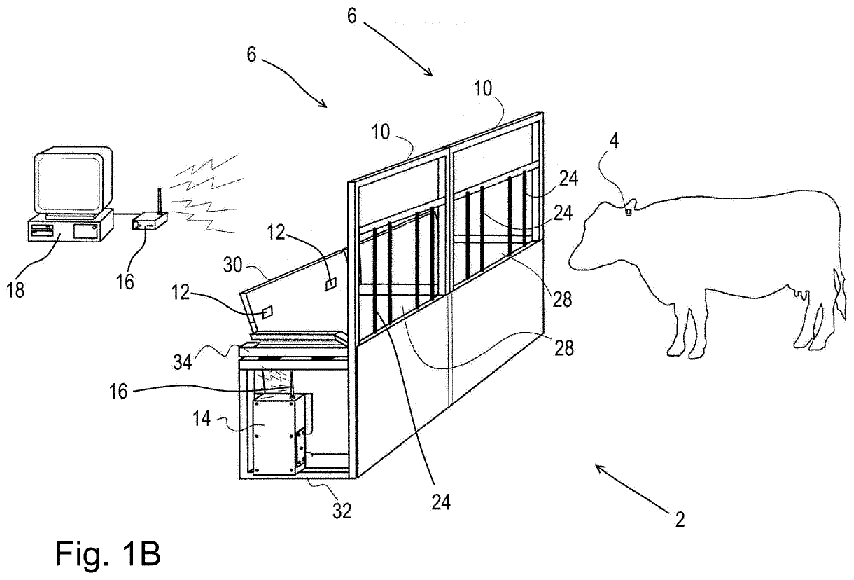

Turning now to FIG. 1, a brief description concerning the various components of the present invention will now be briefly discussed. As can be seen in this embodiment, the system 2 individually identifies an animal by using a transmitter 4 that is generally attached to the particular animal and which identifies the individual animal by a unique animal ID signal.

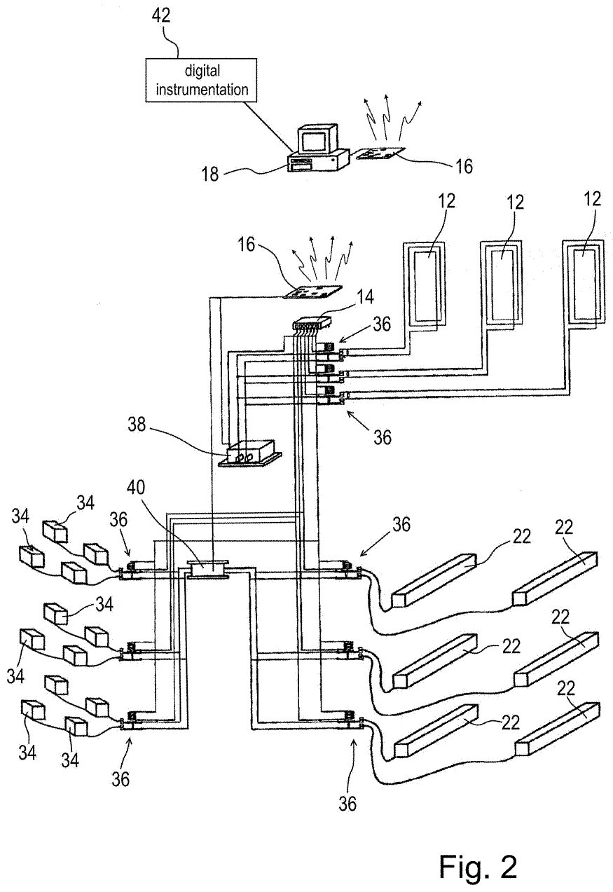

The system 2 further comprises a consumption station 6 having an animal measurement unit 8 which facilitates weighing of the animal when located at the consumption station 6. The term consumption station refers to an arrangement at which one animal at a time can consume feed and/or water. Hereinafter the terms feed station and consumption station can be used interchangeably and generally refer to the same structure. The feed station 6 allows the animal to freely come and go and consume feed based upon the free will of the animal. The feed station 6 includes a front panel 10 having an antenna arrangement 12 which receives the unique animal ID signal from the transmitter 4 attached to the animal. The animal ID signal is passed from the antenna arrangement 12 to a local processor 14 and/or an electronic transmitting and receiving device 16 which transmits the unique animal ID signal to a remote computer 18. The feed station 6 further includes a weight platform 20 having load bars 22 which measure the partial body weight of animals while the animal is consuming feed at the feed station 6. The neck bars 24 and neck guides 26 facilitate positioning of only a single animal on the weight platform 20 at a time. Due to the size of the weight platform 20 and the alignment of the neck bars 24, the animal, during feeding, must insert its head through the opening 28 between the neck bars 24 and place its front legs on the weight platform 20 in order to consume feed from the trough 30 of the feed station 6, which will be discussed in more detail below with reference to FIG. 2. Thus, only the vertical forces exerted by the animal's forelegs are being measured by the load bars 22 associated with the weight platform 20. The antenna arrangement 12 is located on or adjacent the neck bars 24 such that, generally only the antenna arrangement 12 associated with the specific feed station 6 at which the animal is located receives the unique animal ID signal from transmitter 4 of the animal currently feeding at the feed station 6. It is possible however for the antenna arrangement 12 of a feed station 6 to occasionally detect the transmitter 4 of an animal feeding or drinking at an adjacent feed station 6. To minimize the effects of mistaken animal identification, the local processor 14 or remote computer 18 correlates the data analysis to the animal having the relatively greater number of positive identification determinations at the feed station 6.

FIG. 1A illustrates an embodiment of the system 2 having multiple feed stations 6 and associated measurement units 8 for measuring the individual weights of multiple animals. As the system 2 of multiple feed stations 6 is substantially the same as the single feed station 6 described above, only the differences between the two embodiments will be further discussed. It is to be appreciated that due to the neck bars 24 and neck guides 26, only one animal at a time can feed and be weighed at each feed station 6. To facilitate measuring the weights of multiple animals at a time, each feed station 6 comprises its own weight platform 20 and antenna arrangement 12. The antenna arrangements 12 are located such that only the unique animal ID signal transmitted from the transmitter 4 attached to the animal currently feeding from the corresponding feed station 6 can be received by an antenna arrangement 12. All of the antenna arrangements 12 communicate with the local processor 14 and/or the electronic transmitting and receiving device 16 which are mounted to the system 2 of multiple feed stations 6. The local processor 14 and/or electronic transmitting and receiving device 16 receives the unique animal ID signals from the antenna arrangements 12 and then transmits them to the remote computer 18. Each feed station 6 includes a corresponding weight platform 20 having load bars 22 dedicated to that particular feed station 6, such that the partial body weight of only the animal currently located at that particular feed station 6 can be measured while the animal is positioned on the associated weight platform 20. Furthermore, each feed station 6 is associated with a single feed trough 30 that is independent from the feed troughs 30 of adjacent feed stations 6 such that only the animal positioned at a particular feed station 6 can consume feed from the associated feed trough 30. As such, when an animal is positioned at one feed station 6, that animal is incapable of consuming feed from the feed trough 30 of an adjacent feed station 6.

Turning now to FIG. 1B, the feed stations 6 will be described with a focus on the feed troughs 30 thereof. For the sake of clarity, the system 2 of multiple feeding stations 6 is illustrated without a number of the components which are shown located on a front side of the system 2 in FIGS. 1 and 1A. The components missing from the feed stations 6 in FIG. 1B include the weight platform 20 and load bars 22, which facilitate weighing the animal, as well as neck guides 26 which function to position a single animal on the weight platform 20. FIG. 1B illustrates the system 2 of multiple feeding stations 6 including the front panels 10 supported by a base frame 32 which maintain the feed troughs 30 in relation to each other.

The base frame 32 additionally supports the plurality of feed troughs 30 in such a manner so as to permit periodic replenishing of feed. However, the feed troughs 30 should not contact one another as such contact will interfere with determination of accurate weight measurement of the associated feed contained within each respective feed trough 30. The base frame 32 also supports a plurality of load cells 34 which directly support each one of the feed troughs 30 and function as scales. Each one of the feed troughs 30 is supported by one or more load cells 34 which are configured such that the entire weight of each one of the feed troughs 30 and the feed contained therein is focused on and completely supported by the respective one or more load cells 34 for accurately determining the weight of the feed contained within the feed trough 30 at any particular time. The load cells 34 are configured so as to continually monitor and measure the weight of the respective feed trough 30 and transmit such weight measurement signals to the local processor 14 and/or, via the transmission and receiving devicel6, a remote computer 18. It should be noted that the local processor 14 and/or transmission and receiving device 16 are diagrammatically shown in FIG. 1B to be supported on the base frame 32 instead of top of the front panel 10, as shown in FIGS. 1 and 1A. It is to be appreciated that the location of the local processor 14 and/or the transmission and receiving device 16 can vary from one application to another application.

FIG. 2 illustrates the paths via which the identification and weight measurement signals are passed within the system 2. Unique animal ID signals received by the antenna arrangements 12 are relayed, via the switching mechanism 36, to a signal code translator 38 which translates the unique animal ID signal into a unique animal ID code associated with that animal. The local processor 14 sequences the switching mechanisms 36 and the unique code is relayed to the transmitting and receiving device 16. The partial animal weight and feed weight measurement signals can be analog signals that are collected by the load bars 22 and load cells 34, converted into digital weight measurement data by the conversion unit 40 and then relayed to the transmitting and receiving device 16. The transmitting and receiving device 16 transfers weight measurement data and the unique animal ID code to the remote computer 18 for processing.

The local processor 14 and/or transmission and receiving device 16 communicate with the antenna arrangements 12, the load bars 22 of the weight platforms 20 and the load cells 34 supporting the troughs 30. As briefly discussed above, the antenna arrangements 12 continuously receive the unique animal ID signal of the animal currently feeding at the feed trough 30 of the associated feed station 6. It is to be appreciated that when the unique animal ID signal of an animal is received by the local processor 14 and/or the remote computer 18, all the weight measurement data from the load bars 22 of the weight platform 20 and the load cells 34 of the trough 30 are attributed to that particular animal until the time the animal withdraws from and leaves the feed station 6. That is to say, all the weight measurement signals are attributed to the unique animal ID signal until the unique animal ID signal ceases being received by the corresponding antenna arrangement 12 located at the feed station 6.

The local processor 14 and/or the remote computer 16 include a data storage/memory unit (not separately labeled) for recording and storing, at least temporary, the measured and collected weight and unique animal ID signals (codes), from the load bars 22 and cells 34 and the antenna arrangements 12, as well as time signals that correspond to the time at which the weights and information is collected. In the above described manner, a variety of data can be attributed to specific animals and processed by the remote computer 18 to ultimately classify each animal into a specific state which might include healthy, gaining, finished and within these, as in the case of disease, may be able to determine whether an animal is in a state of sub-clinical or clinical disease.

The system 2 and method further comprise digital instrumentation 42 such as digital barometers, thermometers, hygrometers, and rain and snow gauges, just to name a few. These digital instruments 42 as well as other known instruments facilitate measurement, monitoring and recording a variety of environmental conditions such as humidity, temperature, air velocity, barometric pressure and rain/snowfall. U.S. Pat. No. 8,930,148, which disclosure is incorporated herein by reference, indicates that changes in environmental conditions, such as relatively significant temperature changes or changes in the level of humidity can be the cause of inaccurate feed weight measurements or determinations thereof. For example, the weight of feed can either increase or decrease over a period of time based on the amount of moisture absorbed by the feed, during a time period of relatively high humidity, or evaporated from the feed, during a time period of relatively low humidity. An increase in the weight of feed can be especially significant when the feed is exposed to precipitation, such as rain or snow. Inaccurate measurements of feed weight can lead to erroneous feed intake measurements, residual feed intake and feed/water retention determinations. It is to be appreciated that feed intake, residual feed intake and feed/water retention determinations relate to the feed efficiency of an animal, erroneous determinations of these measurements can result in false levels of animal feed efficiency. Although U.S. Pat. No. 8,930,148 may suggest a means of correcting for environmental conditions using a mathematical weighted filtering technique to achieve more accurate feed weight measurements, the system 2 and the method described herein, in contrast, can also consider such environmental conditions when determining and defining behavioral phenotypes of animals.

In the method and the system of determining and defining behavioral phenotypes of animals, the weight measurement data collected with the above described system 2 can include: trough weight data which comprises a trough identifier, a time stamp and weight measurements; body weight data which comprises a scale identifier, a time stamp and body weight measurement, and behavior data which comprises an animal identifier, a time stamp and a location identifier. The location identifier typically relates to the location of the feed trough 30 or rather the feed station 6, but in addition the method and the system, can utilize multiple antennas 12 located within each feed trough 30 (see FIG. 1B). The enables the inventive system 2 to determine the position of the head of the animal while located within the feed trough 30 during feeding. The animal identifier is determined from the unique animal ID signal associated with the identification transmitter 4 attached to the animal. In addition to merely identifying the specific animal, it is also beneficial for additional animal information to be associated with each unique animal ID signal. This additional information can be entered into the method and the system 2 by a computer input device 46 at the time the identification transmitter 4 with its unique animal ID signal is attached to the particular animal. This additional information, to be associated with each animal, can include, for example, the genotype of the animal and one or more known phenotypes or rather physical characteristics, e.g., hide thickness and color, the weight of the animal when the identification transmitter is initially attached such as at the time the animal joins the group of animals. At that same time, it is also beneficial to input information related to any known health issues, medical treatments or procedures of the animal as well as the birth date/age and sex of the animal, such as for example a castrated steer or an in-tact bull.

Another physical characteristic that can be associated with the animal is the physical distribution of the body weight of the animal which may be determined by a body shape analysis. During such body shape analysis, the total body weight is measured, typically by means of a chute and a partial body weight is measured by the automated partial body weight scales, as generally described above. The measurements of the total body weight and the partial body weight of an animal are then utilized to determine a weight factor. The weight factor relates to the body shape of the animal. With reference to FIGS. 16A and 16B, two animals having the same total body weight (chute weight) may have significantly different body weight factors depending body shape of the animal, for example an animal having a larger hind end (FIG. 16A) when compared to an animal having a larger front end (FIG. 16B). Knowledge of a weight factor of a particular animal is beneficial when using the system and the method of the invention. Since the weight platforms 20 of the inventive system 2 only measure the vertical forces exerted by the forelegs of the animal, when the animal feeds at the feed station 6, this partial body weight can be multiplied by the animal's weight factor to determine the total body weight of the animal. Once the body weight factor has been determined, it is not necessary for the animal to pass through a chute in order to measure the animal's total body weight. Also, because the animal will naturally feed at a feed station 6 numerous times each day, a number of weight measurements can be recorded such that small changes in the animal's body weight typically can be observed.

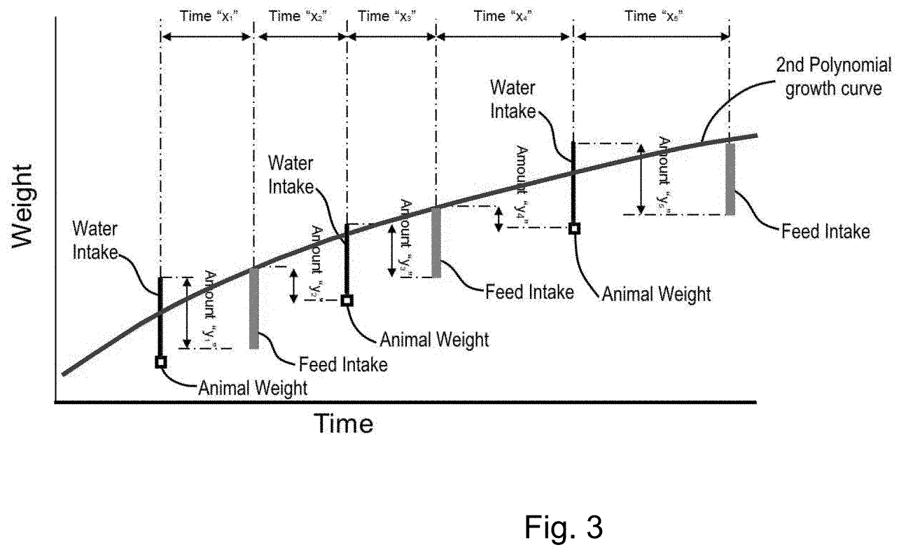

The system and method according to the invention utilizes the measured and collected data for retention modeling that enable feed/water retention is to be utilized as a measure of a feed efficiency of that particular animal or rather a quantification of the desired behavioral phenotypes of the animal. Determining the feed/water retention of an animal can be accomplished utilizing the weight of feed and the weight/amount of the water consumed by the particular animal, i.e., feed intake, water intake, the particular time during which the animal consumes the feed and/or the water, as well as the weight of the animal while at the feed station (see FIG. 3). By plotting changes in the animal's weight and the weight of feed and the weight/amount of the water consumed by the animal during different feeding/drinking events over a period of time, e.g., 24 hrs., one can use a second polynomial growth curve fit in order to determine the average daily gain of the animal over the course of a day.

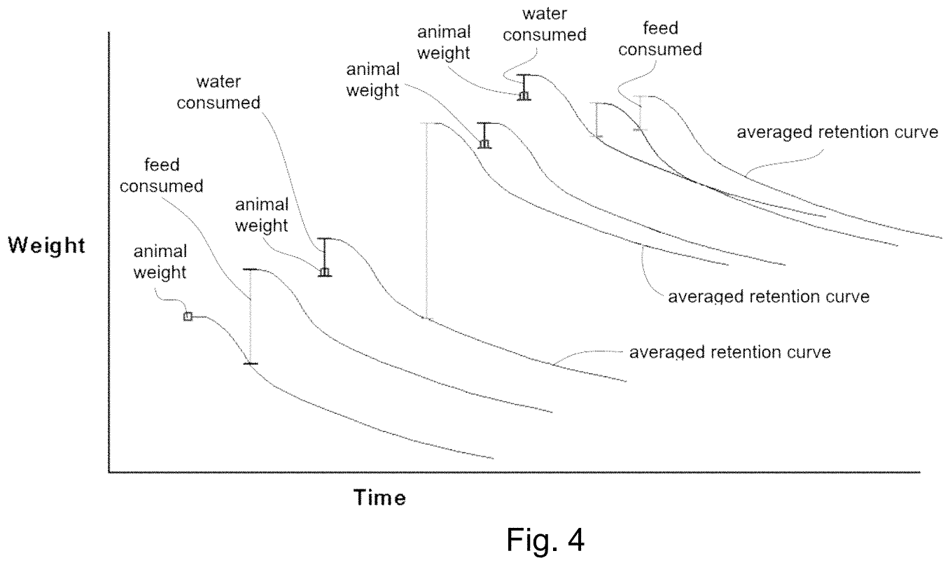

In another example, with reference to FIG. 4, the weight of the animal is measured at the beginning of a drinking event and the weight of the water consumed over the course of the drinking event, e.g., water intake, is measured and/or the weight of feed consumed at the beginning of a feeding event and the weight of the feed consumed over the course of the feeding event, e.g., feed intake, is measured. The animal's weight and the weight of feed or water consumed, during a particular feeding or drinking event, can be plotted in relation to time so as to determine a water/feed retention curve for that particular feeding or drinking event. In FIG. 4, the taller the vertical lines, the more feed or water consumed by the animal. Based on the retention curves determined for a number of feeding and drinking events over a period of time, it is possible to calculate an averaged retention curve for that animal over that time period.

It should also be noted that the inventive system and method enables determination of an individual animal's feed intake and water intake which can be utilized to determine an animal's residual feed intake, average daily gain and average retention curve. The weight of feed and water consumed by an animal can be measured continuously or substantially continuously, e.g., these weight measurements can be collected on a per-second basis and, as such, are hereinafter referred to as “per-second feed intake data”. Two separate data sets are produced from the per-second feed intake data. These two data sets are generally termed “feed events” and “meal events.” The difference between these two data sets is that a feed event occurs on a single feed intake node and a meal event can occur on multiple feed intake nodes with a maximum time allowance between them. For the purpose of the method and the system, only feed events will be considered and further described herein.

The collected per-second feed intake data is natively stored with four pieces of information associated therewith including: the timestamp (the actual time of the event), the unique animal ID signal of the transmitter attached to that particular animal feeding, the weight currently being read, and the location of the feed interval node, i.e., the location of the feed station at which the feed interval occurs. A feed event is defined as a period of time over which the unique animal ID signal of the transmitter being read, without interruption by another animal's unique animal ID signal or a gap in time of over 300 seconds.

Further analysis of the per-second feed intake data can provide other phenotypic information for individual animals. With the system and the method according to the invention, the local processor 14 and/or the remote computer 18, running behavior analysis software, analyzes the collected data and detects additional factors, i.e., time data, which are used to glean further phenotypic information of the individual animals, specifically in relation to an animal's feeding behavior patterns as described below.

According to the disclosure a number of different types of data or rather data sets can be collected and/or measured and used for determining associated parameters. One type of data can generally be referred to as feed data and can include time data, i.e., feeding event start and end times, and weight data, i.e., feeding event start and end weights. These measured and collected data sets can then utilized to determine associated parameters such as feeding event duration, feeding event time, feeding event consumption, feeding rate, feed height, a raw score parameter, a normalized score. The noted data sets are defined and the associated parameters are determined in the manner described in further detail below.

The feeding event start time Tₛₜₐᵣₜ is defined as the time at which the feeding event starts, meaning the time at which a unique animal ID signal is first read at a feeding trough.

The feeding event end time T_{end} is defined as the time at which the feeding event ends, meaning the time at which the unique animal ID signal is last read at the feeding trough.

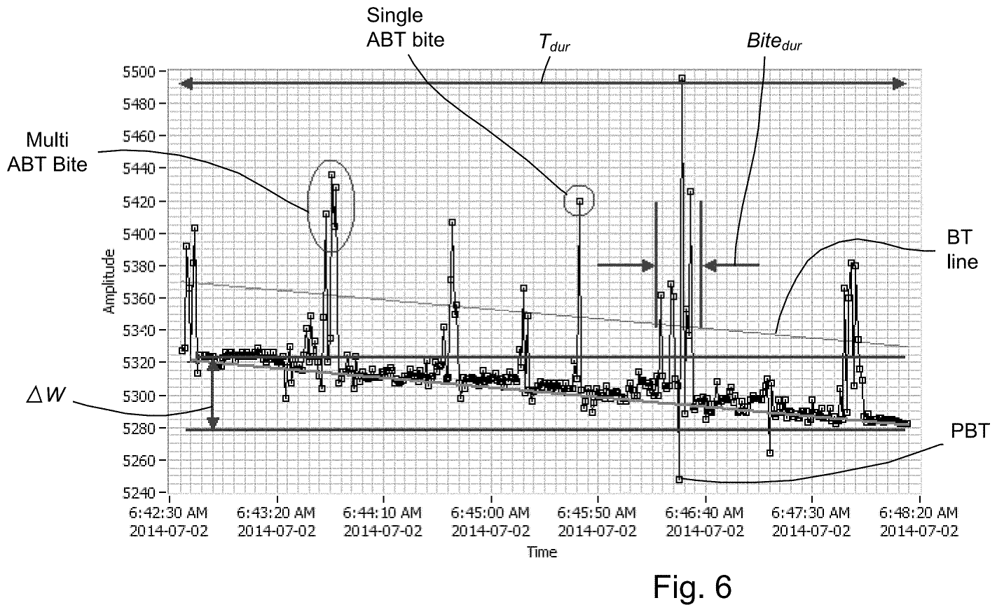

The feeding event duration T_{dur} is defined as the amount of time between the start time and the end time of the unique animal ID signal, as shown in FIG. 6, without any other unique animal ID signal being read therebetween, and the feeding event duration is determined as follows T_{dur}=T_{end}−Tₛₜₐᵣₜ.

The feeding event time T_{FE} is defined as the time at which the feeding event occurred and can be determined as follows T_{FE}=(T_{end}+Tₛₜₐᵣₜ)/2.

To simplify the calculation of the weights, associated with the above time data, bite and animal activity related data is removed from the data by a filter which is applied to the measured weights prior to the further analysis. With the use of the filter, the behavior analysis software further analyses the collected data and detects the associated parameters.

The feeding event start weight Wₛₜₐᵣₜ is defined as the weight of the feed in the feeding trough at the feeding event start time Tₛₜₐᵣₜ.

The feeding event end weight W_{end} is defined as the weight of the feed in the trough at the feeding event end time T_{end}.

The feeding event consumption ΔW, as shown in FIG. 6, is defined as the weight of feed consumed by the animal over the duration of the feeding event duration T_{dur} and is determined as follows: ΔW=Wₛₜₐᵣₜ−W_{end}.

The feeding rate FR is defined as the speed at which the animal consumes the feed over the feeding event duration T_{dur} and is determined as follows: FR=ΔW/T_{dur}.

The feed height Wₐᵥₑ is defined as the average amount of feed in the feeding trough during the feeding event and is determine as follows: Wₐᵥₑ=(Wₛₜₐᵣₜ+W_{end})/2.

The raw score Score is defined as an intermediary parameter for ranking each feeding event and is determined as follows:

Score=(ΔW·Wₐᵥₑ)/10⁶.

The normalized score NormScore, is defined as a range conversion for the scores associated with each of the load cells 34 of the feed troughs 30 at which the animal consumed feed over a period of time, e.g., 24 hrs and is determined as follows: NormScoreᵢ=(Scoreᵢ−min(Score))/(max(Score)−min(Score)).

From the above data and associated parameters a feeding hierarchy rank

Rank of the individual animals in the group of animals can be determined. Feeding hierarchy rank Rank is considered to be a measure of the animals social rank within the group of animals and correlates to the order in which the animals in the group feed at the feed stations, generally an animal with a high feeding hierarchy rank Rank will feed before an animal with a lower feeding hierarchy rank Rank. To determine the feeding hierarchy rank Rank an average NormScore value can be calculated for each animal (unique animal ID signal) on each weighing scale at which the animal consumes feed from the NormScore values of all the feeding events registered to that unique animal ID signal and the rankings of these average NormScore values are then averaged, across all weighing scales, to determine the overall feeding hierarchy rank Rank for each of the animals in the group.

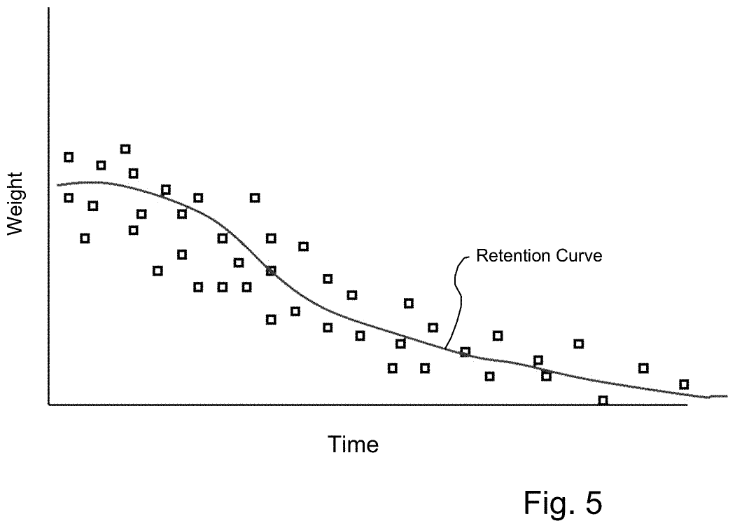

As shown in FIGS. 3-6, the behavior analysis software runs a linear regression on the filtered weight-time data for each feeding event to establish a Baseline Feed Disappearance line (BFD) and a standard deviation of the feeding event (σ_{f}) for raw data less the BFD. Further, an offset line is defined 2×σ_{f} above the BFD and is called the Bite Threshold (BT). Unfiltered data points, during the feeding event occurring above the BT line, are logged as Above Bite Threshold events (ABT). FIG. 6 highlights a Single ABT bite event as well as a Multi ABT bite event. Consecutive ABTs and ABTs with four or fewer data points below the BT between them are grouped into a single Bite event (bite). The data points within a single bite that are between ABTs are known as Proxy Bite Threshold events (PBT) and an example of which is shown in FIG. 6. More than two data points below the BT determines a separation between bites. Further analysis of the data by the behavior analysis software detects a bite frequency and an average bite duration.

The bite frequency bite_{freq} which is defined as the total number of bites per unit time of the feeding event and is determined as follows

bite_{freq}=Σbite/T_{dur}

The average bite duration bite_{dur} (see FIG. 6) which is defined as the mean number of samples collected per bite and is determined as follows:

bite_{dur}=(ΣABT+ΣPBT)/(Σbite).

Using the system and method according to the disclosure including behavior analysis software, termed as Process Who Eats First.vi and developed by the Applicant, a number of data points, as diagrammatically shown in FIG. 6, can be determined and stored in the inventive system including: Rank, NumFE, AveDur, AveFI, BiteFreq, St_Dev, Pts_St_Dev, BiteDurat and ConsecHits, each of which are described below in further detail.

Rank, as described above, relates to the Feeding Hierarchy Rank using a simple average across the bunks to determine the average rank value, which implies that two animals could have the same rank in a trial.

NumFE is defined as the total number of feeding events stored in an animal's file within a memory unit which communicates with the processor.

AvgDur is defined as a simple average of the feeding event duration (T_{dur}) of all feeding events for an animal and is stored as total number of seconds within the memory unit.

AvgFI is defined as a simple average of the consumption (ΔW) of all of the individual feeding events for an animal and is stored as a total number of grams within the memory unit.

BiteFreq is defined as the Bite frequency described above, averaged over all feeding events for an animal and is stored as a total number of bites per second within the memory unit.

St_Dev is defined as the standard deviation of a feeding event (σ_{f}) noted above and calculated by subtracting the BT line from raw bite force data and calculating a standard deviation of the result and is stored in total number of grams and averaged across all feeding events for each monitored animal.

Pts_St_Dev is defined as the number of data points above the BT line (2×σ_{f}) and is taken as a simple average across all feeding events for each monitored animal.

BiteDurat is defined as a simple average of the average bite duration bite_{dur} of all feeding events for each monitored animal and is stored as a total number of seconds within the data storage/memory unit.

ConsecHits is a tally of data-points where an animal maintains force above the bite threshold with four or fewer seconds in between, and is used in calculating bite_{dur} and calculated as a simple average for all feeding events for each monitored animal.

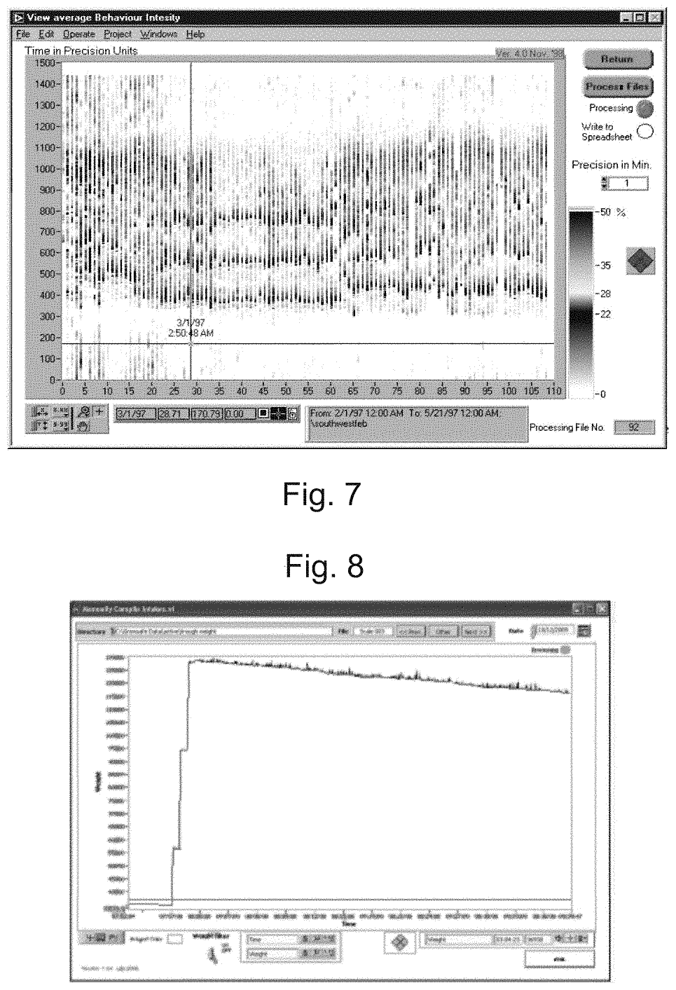

The inventive method and system can be utilized to collect and analyze data which assists with identifying and defining animal behavioral phenotypes, i.e., traits that until now have not been accurately specified or defined. The above collected weight data and behavior data, as well as data from other data sources, can be analyzed so as to define a number of behavioral phenotypes. The behavior analysis software enables the determination of “who feeds first” and order indexing in which the order is normalized by ranking and an order number is assigned. Also order/quantity can be indexed in which quantity is multiplied by the inverse of the order. The unit of quantity is determined as being equal to the total feed consumed between feed supply event divided by the number of animals available, again order is normalized by ranking and an order number is assigned. FIGS. 7 and 8 are diagrammatic screen captures of the behavior analysis software as it is utilized by the inventive method and system in the determination of who feeds first.

The order of who feeds first can be linked to certain behavioral phenotypes and the state of the animal. One behavioral phenotype associated with who feeds first is the aggression level of the animal based on the recognition that more aggressive animals will push less aggressive animals away from the feed station during a time period of high animal traffic. Another trait that can be more accurately identified and defined, with the knowledge of who feeds first and associated with aggressive behavior, is an animal's residual feed intake. More aggressive animals have been found to spend more time and energy defending their territory thereby reducing the animal's residual feed intake. The order of who feeds first can also correspond to an animal's health and robustness since unhealthy animals will not waste energy fighting for territory and non-robust animals will never be at the high end of the dominance order. The order of who feeds first is also a good indicator of social dominance as dominant animals will normally feed first.

The behavior analysis software enables the determination and analysis of an animal's consistency of feeding which can include the determination of the variation of an animals feeding behavior, on a day to day basis, which is measured as the standard deviation in kilograms of daily feed intake of the monitored animal. The consistency of feeding also includes the determination of the variation of an animals feeding behavior on an hour to hour basis throughout the day. In the determination of variation over the time period of a day, the day can be compartmentalized in a variable number of even or user selectable sections. Feed intake, for every section for every day, is calculated and the variation over a trial period is expressed in standard deviations. The variation throughout the day is expressed by the average of all the sections in kilograms.

The inventors have determined that an animal's consistency of feeding can be an indicator of acidosis as more consistent feeding reduces digestive upset. Feeding consistency is also an indication of an animal's residual feed intake in that more consistent feed enhances the feed efficiency of an animal. An animal's Average Daily Gain (ADG) corresponds to the feeding consistency of the animal because more consistent feed promotes animal growth. Further, an animal having a more consistent feeding behavior is better equipped to do well under varying circumstances, this being a measure of the animal's robustness. The inventors have determined that feed consistency can further be an indicator of liver abscesses and other sicknesses due to the fact that acidosis causes liver failure and can compromise the animal's immune system.

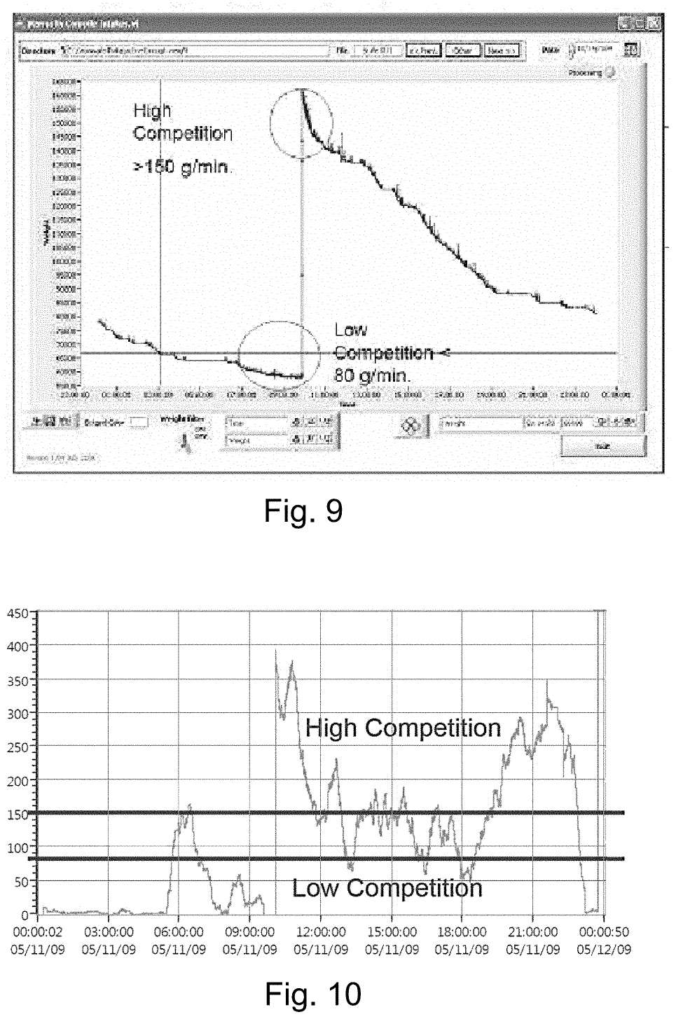

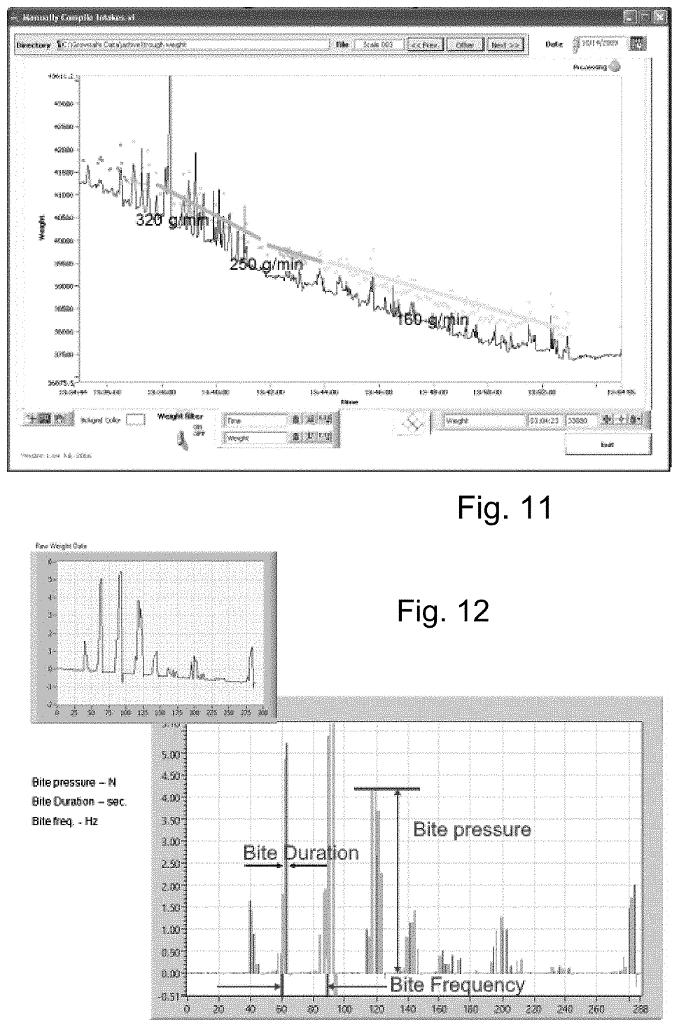

FIGS. 9, 10 and 11 are diagrammatic screen captures of the behavior analysis software of the invention as the software is utilized by the inventive system in the analysis of feeding rate FR. As shown in FIGS. 9 and 10, the feeding rate FR of an animal can vary depending on the number of other animals in the group. When competition for feed is high, an animal tends to feed at a quicker rate so as to consume a greater amount of food over a shorter amount of feeding time due to the concern that a more dominant animal will take over the feed station. When the competition for feed is low, the animal tends to feed at a slower rate as being displaced from the feed station by more dominant animals is less likely to occur. FIG. 11 is a screen capture that illustrates the analysis of feeding rate FR of feed events for three animals. As shown in FIG. 11 a first animal feeds at a rate of 320 g/min, while a second animal feeds at a rate of 250 g/min and a third animal feeds at a rate of 160 g/min. From FIG. 11 one could conclude that the third animal is more dominant than the first and the second animals, while the second animal is more dominant than the first animal.

The behavior analysis software can also be utilized, by the inventive system, to determine and analyze an animal's bite size while feeding during a feeding event. As shown in the diagrammatic screen capture illustrated in FIG. 12, it is possible, by the behavior analysis software, to consider or determine bite size, bite frequency, bite duration and bite pressure of each monitored animal during each feeding event.

Further, the behavior analysis software analyzes the collected data to determine an animal's ranking related to empty bunk attendance. FIG. 13 illustrates a diagrammatic screen capture, of the behavior analysis software, during determination of empty bunk attendance ranking. Empty bunk attendance is understood as an animal's presence at a feed trough 30 when the feed trough is empty. In the graph of FIG. 13, the presence of the animal at the trough is shown by the dots extending over a period of time. The spikes in the weight measurements represent pressure applied to the trough by the animal, for example by the animal licking the surface of the empty trough. It is noted that the relative consistent weight of the trough before and after the presence of the animal, is indicative of the fact that the animal consumed no feed. The smaller spikes in the weight both before and after the presence of the animal are representative of signal noise, wind or the like.

Another feeding pattern or behavior of an animal that can be analyzed, by the behavior analysis software, relates to feed sorting as shown in the diagrammatic screen captures illustrated in FIG. 14. That is, the data collected by the inventive system can be analyzed to determine the feed sorting behavior of an animal and thus the overall health of an animal. In the graph of FIG. 14, the presence of the animal at a feed trough 30 is shown by the dots extending over a period of time. The lack of spikes in the weight measurements is representative of minimal or no pressure being applied to the feed by the animal. This behavior can occur after the animal is finished consuming feed. Relatively larger spikes during such behavior are indicative of an animal consuming “desired” feed particles after digging through other “less desired” feed particles.

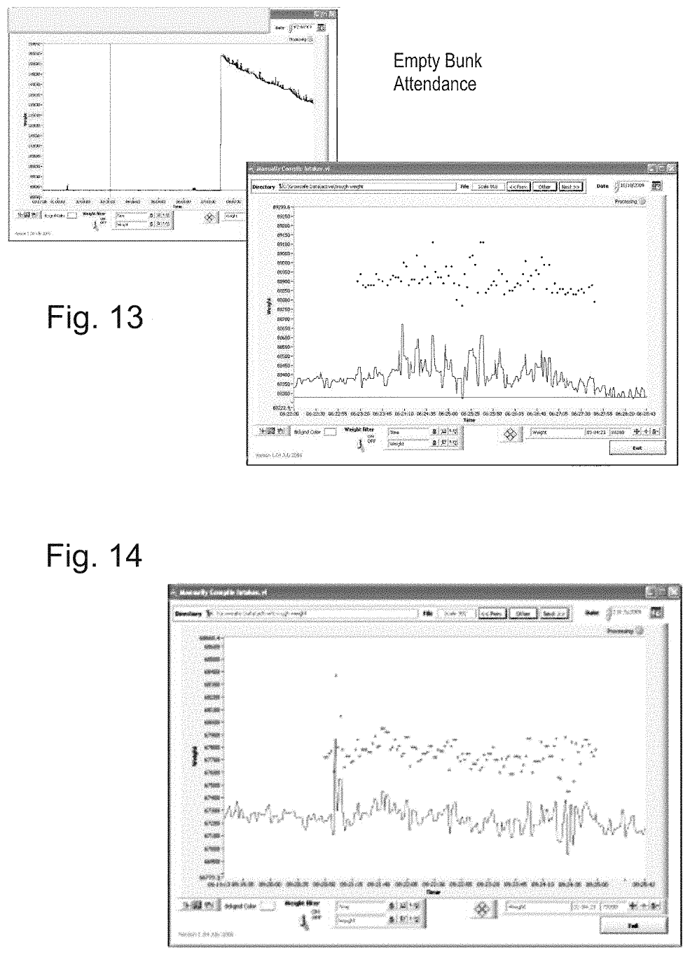

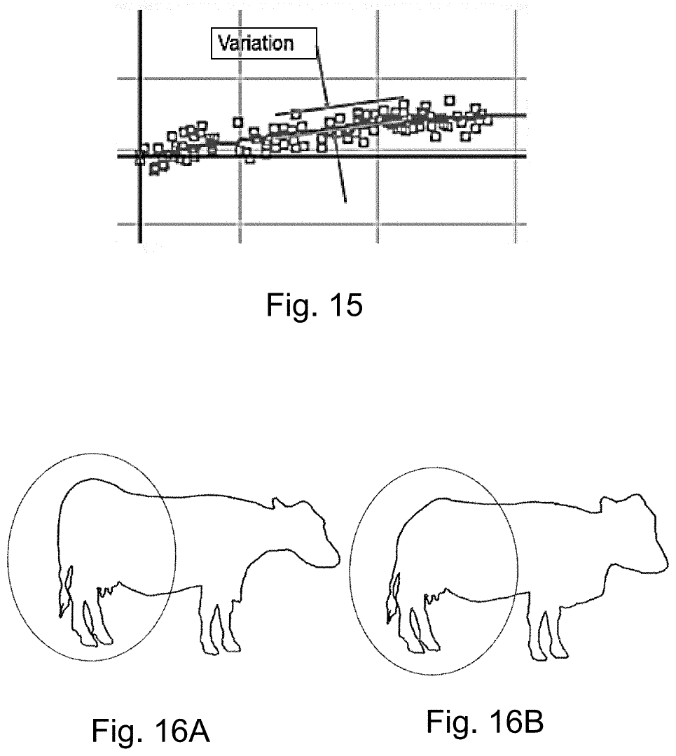

The inventors have determined that a still further trait of animals, termed as “flightiness,” can be determined by analysis of the collected data. The screen capture of the behavior analysis software, as diagrammatically shown in FIG. 15, illustrates variations in the front end weights of an animal. It is believed an animal that is “less flighty” is more docile and thus burns less energy than an animal which is deemed “more flighty.” The front end weights of an animal are measured over a period of time and the standard deviation of the weights is a measure of an animal's flightiness. An average of the worst 10% of the measured weight values collected is an indicator of flightiness max weight.

With the ability to specifically and accurately define a large number of behavioral phenotypes to determine a variety of particular behavioral patterns of the individual animals, it is thus possible to identify animal behavioral phenotypes that correlate with animals that have a high production yield. That is to say, particular behavioral phenotypes have been found to correspond to animals having the desirable attributes of a healthy, successful animal such as animals that have a high feedlot performance, feed efficiency rating, average daily gain or animals that rarely need medical intervention.

It is to be appreciated that the altitude, at which the cattle is being raised is initially inputted into the system and the method as a fixed parameter. In addition, a subjective indication of the type of grazing/raising terrain, e.g., whether the grazing/raising terrain is relatively flat, has small undulations or rolling hills, relatively hilly, mountainous, etc., for raising the cattle is initially inputted into the system and the method as another fixed parameter. Further, the amount and the type of the local vegetation contained on the grazing/raising terrain are initially inputted into the system and the method as a further fixed parameter.

In addition to these fixed parameters, the system and the method, according to the invention, also continuously collect local environmental data at the same time that the drinking and feeding events are being gathered by the system and the method. That is, numerous times each day, the system and method will record the current environmental conditions, such as, the current temperature, the current wind speed and/or direction, the current humidity, the current barometric pressure, etc. The inventors enters have determined that local environmental conditions can have a significant impact on which genotype(s) and/or phenotype(s) will thrive and which will not. The system and the method, according to the invention, is particularly useful in identifying the particular genotype(s) and/or phenotype(s) that thrive under the local environmental conditions, and this information can be particularly useful in assisting cattle ranches, which have similar local environmental conditions, with acquiring new cattle to raise on their respective ranches to improve cattle output while utilizing a minimal amount of feed.

The inventors have determined that having the ability to identified a particular behavioral phenotype(s), e.g., animals that have a high feedlot performance, feed efficiency rating, average daily gain and/or animals that rarely need medical intervention, has a variety of advantages. In particular, this information can be utilized by cattle ranchers when either breading cattle to be raises on their cattle ranch or when acquiring new cattle from (local) breeders to be raised on their cattle ranch. That is, the cattle ranchers can, based upon the identity of preferred phenotype(s) and preferred genotype(s), use this information to either breed or acquire new cattle for raising on their cattle ranch. By appropriate selection of the preferred phenotype(s) and/or preferred genotype(s) of the animals to be raised on a particular cattle ranch, a cattle rancher can maximize the cattle output from the cattle ranch while minimizing the feed and watering expenditures associated with raising such cattle.

Further, the same or similar information can be utilized by local breeders in determining which type of cattle to be breed, i.e., breeding cattle having the preferred phenotype(s) and preferred genotype(s) for the local terrain, local altitude and local environment conditions, e.g., flat terrain, hilly terrain or mountainous terrain; a hot environment, moderate environment or a cold environment; a dry environment, moderate environment or a humid environment; sea level, moderate altitude or a high altitude; etc. The inventors have determined that while one particular breed of cattle may grow particularly well on certain terrain, at a particular altitude and under particular environment conditions, this does not necessarily mean that the same breed of cattle will grow well on different terrain, and/or at a different altitude and/or under different environment conditions. The present system and method are directed at evaluating/determining/identifying the particular phenotype(s) and genotype(s) of animals which will grow most efficiently in view of the local terrain, local altitude and the local environment conditions.

The computer readable medium as described herein can be a data storage device, or unit such as a magnetic disk, magneto-optical disk, an optical disk, or a flash drive. Further, it will be appreciated that the term “memory” herein is intended to include various types of suitable data storage media, whether permanent or temporary, such as transitory electronic memories, non-transitory computer-readable medium and/or computer-writable medium.

It will be appreciated from the above that the invention may be implemented as computer software, which may be supplied on a storage medium or via a transmission medium such as a local-area network or a wide-area network, such as the Internet. It is to be further understood that, because some of the constituent system components and method steps depicted in the accompanying Figures can be implemented in software, the actual connections between the systems components (or the process steps) may differ depending upon the manner in which the present invention is programmed. Given the teachings of the present invention provided herein, one of ordinary skill in the related art will be able to contemplate these and similar implementations or configurations of the present invention.

It is to be understood that the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

While various embodiments of the present invention have been described in detail, it is apparent that various modifications and alterations of those embodiments will occur to and be readily apparent to those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having,” and variations thereof herein, is meant to encompass the items listed thereafter and equivalents thereof as well as additional items while only the terms “consisting of” and “consisting only of” are to be construed in a limitative sense.

The foregoing description of the embodiments of the present disclosure has been presented for the purposes of illustration and description. It is not intended to be exhaustive or to limit the present disclosure to the precise form disclosed. Many modifications and variations are possible in light of this disclosure. It is intended that the scope of the present disclosure be limited not by this detailed description, but rather by the claims appended hereto.

A number of implementations have been described. Nevertheless, it will be understood that various modifications may be made without departing from the scope of the disclosure. Although operations are depicted in the drawings in a particular order, this should not be understood as requiring that such operations be performed in the particular order shown or in sequential order, or that all illustrated operations be performed, to achieve desirable results.

1. A method of optimizing production profitability at an animal production facility by genetic selection based upon behavioral phenotypes, the method comprising:
providing a plurality of consumption stations at the animal production facility;
assigning each animal of a herd with an unique identification detection device;
detecting, via the unique identification detection devices, each time any animal of the herd is one of feeding or drinking at one of the plurality of consumption stations;
collecting, via at least one body weight sensor respectively associated with each one of the plurality of consumption stations, high frequency data relating to a body weight of each animal feeding or drinking at one of the plurality of consumption stations;
collecting, via at least one feed weight sensor respectively associated with each one of the plurality of consumption stations, high frequency data relating to at least one of an amount of feed being consumed by each animal feeding at one of the plurality of consumption, a bite size, a bite frequency, a bite duration and a bite pressure of each animal feeding at one of the plurality of consumption stations;
analyzing, with a processor, the collected high frequency data relating to the body weight of each animal, the collected high frequency data relating to the at least one of the amount of feed being consumed by each animal, the bite size, the bite frequency, the bite duration and the bite pressure of each feeding animal to determine at least one desired behavioral phenotype which optimizes production profitability; and
either selectively introducing new animals having the at least one desired behavioral phenotype into the herd or selectively breeding the animals of the herd having the at least one desired behavioral phenotype to increase a population of animals in the herd having the at least one desired behavioral phenotype for optimizing the production profitability of the production facility.

2. The method according to claim 1, further comprising identifying, via the processor, animals of the herd having the desired behavioral phenotypes and selectively breeding the identified animals

3. The method according to claim 1, further comprising collecting the high frequency data relating to the body weight, and the high frequency data relating to the at least one of the feed being consumed, the bite size, the bite frequency, the bite duration and the bite pressure of each feeding animal at a rate of at least one sample every 10 to 20 seconds or less.

4. The method according to claim 1, further comprising collecting the high frequency data relating to the body weight, the high frequency data relating to the at least one of feed being consumed, the bite size, the bite frequency, the bite duration and the bite pressure of each feeding animal at a rate of at least a plurality of samples every second.

5. The method according to claim 1, further comprising reproducing new animals for the herd by mating a bull, which has the identified desired behavioral phenotypes, with cows of the herd which have the identified desired behavioral phenotypes so as to reproduce offspring with the identified desired behavioral phenotypes.

6. The method according to claim 1, further comprising reproducing new animals for the herd by collecting at least one of sperm from a bull, which has identified desired behavioral phenotypes, and embryos from one or more cows, which have the identified desired behavioral phenotypes, and fertilizing the collected embryos with the collect sperm to produce fertilized embryos, and implanting the fertilized embryos in cows of the herd to create offspring with the identified desired behavioral phenotypes.

7. The method according to claim 1, further comprising correlating data regarding at least one environmental condition that corresponds to the animal production facility to the at least one desired behavioral phenotype; and
selectively introducing new animals or selectively breeding the animals having the at least one desired behavioral phenotype based on the at least one environmental condition corresponding to the animal production facility.

8. The method according to claim 1, further comprising introducing animals having at least one desired behavioral phenotype, that is positively associated with at least one environmental condition, into an animal herd at a different animal production facility having substantially the same at least one environmental condition.

9. A method for defining animal behavioral phenotypes which, in a specific environment, are beneficial for the physiology of an animal located within the specific environment, the method comprising the steps of:
collecting consumption data and weight gain data for the animal over a period of time;
collecting animal behavioral data of the animal over the period of time;
analyzing and manipulating the consumption data, the weight gain data and the behavioral data of the animal to define a positive behavioral phenotype that correlates to a high state of the physiology of the animal that is greater than the physiology of an animal not possessing the positive behavioral phenotype; and
selectively breeding the animals possessing the positive behavioral phenotype to produce animals having a physiology that is greater than a group of animals not possessing the positive behavioral phenotype.

10. A system utilized for optimizing production profitability at an animal production facility by genetic selection based upon behavioral phenotypes, the system comprising:
a plurality of unique identification transmitters, each of the plurality of identification transmitters being assigned and attached to a corresponding particular animal of an animal herd at the animal production facility, the identification transmitter identifying the corresponding animal by a unique signal;
a plurality of consumption stations at the animal production facility at which the animals of the animal herd consume feed or water, each of the plurality of consumption stations having:
an antenna arrangement which detects, via the unique signal, each time any animal of the herd is consuming the feed or the water at the consumption station,
animal weight measurement unit which collects high frequency data relating to a body weight of the animal consuming the feed or the water at the consumption station;
at least one feed weight load cell which collects high frequency data relating to at least one of an amount of the feed or the water being consumed by the animal at the consumption station, a bite size, a bite frequency, a bite duration and a bite pressure of each animal feeding at the consumption station;
a processor communicating with the antenna arrangement, the animal weight measurement unit and the at least one feed weight load cell of each of the plurality of consumption stations, the processor receives and analyzes the collected high frequency data, from each of the plurality of consumption stations, relating to the body weight of each animal, the collected high frequency data, from each of the plurality of consumption stations, relating to the at least one of the amount of feed being consumed by each animal, the bite size, the bite frequency, the bite duration and the bite pressure of each feeding animal to determine at least one desired behavioral phenotype which optimizes production profitability; and
the system identifying, based upon the high frequency data, the at least one desired behavioral phenotype of the animals of the herd to assist an operator of the animal production facility with acquiring new animals having the at least one desired behavioral phenotype so as to optimize the production profitability of the animal production facility.
